(19) United States
(12) Patent Application Publication
(10) Pub. No.: US 2002/0115190 A1
(43) Pub. Date: Aug. 22, 2002

(54) Culture of staphylococcus aureus and a method for preparing the same

(76) Inventor: Juyu Chen, Shenyang (CN)

Correspondence Address: DANA L. TANGREN, WORKMAN NYDEGGER & SEELEY, 1000 EAGLE GATE TOWER, 60 EAST SOUTH TEMPLE, SALT LAKE CITY, UT, 84111, US

(21) Appl. No.: 10/073,681

(22) Filed: Feb. 11, 2002

(30) Foreign Application Priority Data
Feb. 16, 2001 (CN) 01104103.X

(51) Int. Cl. C12N 1/20; C12N 1/12; C12N 1/00

(52) U.S. Cl. 435/252.1; 435/883

(57) ABSTRACT

A method for preparing a culture of Staphylococcus aureus includes adding pork heart into water and smashing to obtain an extract of pork heart. Peptone and NaCl are then added to the extract to obtain a medium. A strain of S. aureus CGMCC 0485 is recovered and proliferated to obtain a seed solution. The seed solution is combined with the medium and then fermented to obtain a culture, the culture having an anticancer effect.

### CROSS-REFERENCE TO RELATED APPLICATIONS

This application claims priority to China Patent Application No. 01104103.X, filed, Feb. 16, 2001.

### BACKGROUND OF THE INVENTION

1. The Field of the Invention

The present invention relates to a culture of Staphylococcus aureus with anticancer effect and a method for preparing the same.

2. The Relevant Technology

The cultures of Staphylococcus aureus contain the anticancer components, which is staphylococcal enterotoxin (SE) contained in the mainly effective components. The toxin is considered as a kind of superantigen (SAg) with strong biological activity at present. The sources of superantigens include bacterium, virus and parasite etc. according to present references. The meaning, action and the mechanism of action of superantigens are different from that of all the present common antigens, i.e., conventional antigens. Superantigen is a kind of protein which has a complex source. Only a trace of superantigen is needed in immune response, for example, it can be counted according to ng. This kind of antigen can stimulate the mass proliferation of T cell by a kind of very special mechanism, and produce a great deal of cellular factors. Its expressive cytotoxicities are the following: (1) stimulating the T lymphocytes with cytotoxicity directly, and making them produce the killer effect to target cells; (2) stimulating NK cells by the effects of cellular factors; (3) killing the cancer cells by the superantigen dependent cell-mediated cytotoxicity (SDCC).

In recent years, the chemical method was used to synthesize McAb-SEA compound after it was testified that superantigen had the anticancer effect, and then the recombinant technique was used to produce the fusion protein. Phase I clinic test was conducted with the fusion protein in 1997. The result showed that it was safe when the dosage was 1.5 ng/kg, but the overall reaction of patients was rather strong. The amount of the leucocytes of most patients receiving treatment was distinctly reduced, and the amount of blood platelet was reduced more.

### SUMMARY OF THE INVENTION

In order to overcome the problem in the art, the object of the present invention is to provide a new strain of S. aureus.

The second object of the present invention is to provide a type of culture of S. aureus, its culture method and the medium used in culture.

The third object of the present invention is to provide the anticancer effect provided by the aforementioned culture of S. aureus.

A culture is prepared with the strain of S. aureus by fermenting the cells in a new medium according to the present invention, in which its culture has the anticancer effect. The formulation of the medium according to the method of the present invention is simple, which can reduce the amount of raw material effectively. It is not needed to sterilize for the filtrate of the culture used in treatment. The stability of the enzyme produced by the strain is increased. The amount of the leucocytes of the patients increases greatly and the anticancer effect is improved after using the product.

The strain of Staphylococcus aureus of the present invention was deposited at the China General Microorganism Collection Center (CGMCC) on Sep. 14, 2000, under the accession number of CGMCC 0485.

The strain CGMCC No.0485 is obtained by mutating with nitrosoguanidine from CGMCC No. 0165, which is obtained by screening from more than 100 strains of S. aureus obtained by isolating from the specimens in the hospital clinic bacterium inspection labs.

Morphology of Strain CGMCC No.0485:

Morphological Characteristics:

Standard strain of Staphylococcus aureus and the identifying cells were magnified 20,000 times (embed method) and 60,000 times (ultra-thin section method) by a transmission electron microscope. The cells are globular, diameters are 0.5-1.0 μ m, single, arranging in pairs, charactered in more than one plane divisions, forming the irregular groups, no flagella, no motility, no capsule, and no endospores. The cell wall, the cell membrane and the chromatin can be observed by a transmission electron microscope (ultra-thin section method). In general, the size and structure of cells are different in different growth stages, but there is no distinct difference between Standard strain of Staphylococcus aureus and the identifying cells.

Culture Characteristics:

Culturing on blood plates, the clones are round, protuberant, smooth-faced, brim-regular, opaque, golden colorful, and have big and transparent hemolytic loops. When cultured at 35° C. for 24 hours, the diameter of clone is about 1.5-2.0 mm.

The culture is turbid in liquid medium at first, then becomes clear, and has thin and suspending precipitation. There is a ring shaped film when cultured for 2-3 days. The turbidity of liquid culture is slightly little and the concentration of cells is a little low under the same conditions.

Strain Characteristics:

The Standard strain of Staphylococcus aureus and the identifying cells are stained with standard gram stain. The results showed that Standard strain of Staphylococcus aureus and the identifying cells were both gram-positive bacteria by observing in a microscope.

The Standard strain of Staphylococcus aureus and the identifying cells are stained with capsule stain. The results showed that Standard strain of Staphylococcus aureus and the identifying cells were negative by observing in a microscope.

The Standard strain of Staphylococcus aureus and the identifying cells are stained with spore stain. The results showed that Standard strain of Staphylococcus aureus and the identifying cells were negative by observing in a microscope.

Metabolism Characteristics:

Test of blood coagulase is positive, and the ability of producing enzyme is very strong in the medium.

Tests of glucose fermentation and manitol fermentation were performed, and the ability of producing enzyme is a little weak.

The eligible rate of heat source of the filtrate after the culture is filtered is high, which can be above 95%.

Test of Starch Hydrolysis: Standard strain of Staphylococcus aureus and the identifying cells are both negative.

Catalase Test: Standard strain of Staphylococcus aureus and the identifying cells are both positive.

The preparation method of the present invention comprise the following steps:

(1) Adding the pork heart into 1-2 volume water, smashing, filtering and obtaining the first filtrate and residue;

(2) Adding 1-2 volume clean water of 90-95° C. into the said residue, soaking, smashing and obtaining the second filtrate and residue; Combining the first and second filtrates to obtain the third filtrate;

(3) To the combined third filtrate, adding 0.025-0.5 g peptone and 0.30.9 g NaCl based on 100 ml resulting medium, Then adding activated carbon, and adjusting pH from 8.5 to about 7.2 gradually to obtain resulting medium;

(4) Recovering and proliferating the strain of Staphylococcus aureus CGMCC 0485, and preparing the seed solution, inoculating it to the medium when its bacterium concentration is 105-107, and the amount of inoculation is 0.02-0.2 ml based on 100 ml resulting medium;

(5) Culturing the strain CGMCC 0485 at about 35° C. for about 15-20 hrs to obtain the culture;

(6) Sterilizing and filtering the said culture, adjusting the osmotic pressure to isotonic and pH to 6.8-7.4.

The product of the present invention can be processed directly, filling and enveloping it to make injection for intramuscular injection. The dosage of the injection to tumor patients is: one injection per day and 2 ml per injection.

The strain of Staphylococcus aureus in the aforementioned method was deposited at the China General Microorganism Collection Center (CGMCC) on Sep. 14, 2000, under the accession number of CGMCC 0485.

⅕ of fermentation time is shortened distinctly with the strain in the present invention. And ⅔ of the culture time of seed solution is shortened. The use of materials is reduced. The technique is simple and can be controlled easily. The quality of product is increased and the cost is reduced. In addition, the ability of producing enzyme of the strain increases greatly after mutation. The need for nutrition condition of the strain is low. The qualification rate of pyrogen of the product increases greatly, and the side reaction is reduced.

### DETAILED DESCRIPTION OF THE PREFERRED EMBODIMENTS

In order to further understand the present invention, it is more specifically explained by the following Examples and Experimental Examples, but is not limited thereby.

### EXAMPLE 1

Take 100 kg pork heart and wash it with water, then smash it by smashing machine (produced in Meat and food machine company in Fanyu, Guangdong province, Type: TJL12-4). Add 150 kg injection-water and soak for 1 hr at 90° C., then filter to obtain a filtrate of the pork heart and a residue.

Add 100 kg injection-water into the aforementioned residue, soak at 90° C. for 1 hr, filter and discard the residue, and obtain the filtrate. Combine all the filtrates. To 2000 ml combined filtrate of pork heart, add 100 g peptone and 1200 g NaCl, then dissolve all the added materials by stirring and boiling. Add the clean water again, and adjust its pH to about 8.5, then keep it at 4° C. overnight. Add 10 g active carbon to the filtrate, and adjust pH of the aforementioned filtrate from 8.5 to about 7.2 gradually.

Adjust aforementioned treated filtrate to be isotonic, and fill it into the 500 ml sealed flasks, then sterilize for 20 minutes at 0.1 MPa to obtain 400 kg medium. Its ; z total volume is about 400,000 ml.

Recover the strain of S. aureus CGMCC 0485 at 35° C. for 24 hrs, then w z Q i a proliferate cells on the blood plates at 35° C. for 8 hrs to obtain the seed solution. The bacterium concentration of the seed solution is 107.

Then, mix the medium in a stainless steel vessel and put it into a fermenter after sterilizing and filtering. Inoculate it after preheating at 35° C. The amount of inoculation is 0.2 ml per 100 ml medium. Ferment it at 35° C. for 16 hrs to obtain the culture. Fill it to the ampoule after the said culture is sterilized, filtered and examined eligibly.

Each culture obtained in fermentation is tested by pyrogen, enzyme activity and hypersusceptibility.

### EXAMPLE 2

Take the pork heart 100 kg and wash it with water, then smash it by smashing machine (produced in Meat and food machine company in Fanyu, Guangdong province, Type: TJL12-4), add 150 kg injection-water and soak for 1 hr at 90° C., then filter to obtain a filtrate of the pork heart and a residue.

Add 100 kg injection-water into the aforementioned residue and soak at 90° C. for 1 hr, then filter and discard the residue to obtain the filtrate. Combine all the filtrates. To 40000 ml combined pork heart filtrate, add 2000 g peptone and 3600 g NaCl. Dissolve all the added materials by stirring and boiling. Add the clean water again and adjust its pH to about 8.5, then keep it at 4° C. overnight. Add 200 g active carbon to the filtrate and adjust pH of the aforementioned filtrate from 8.0 to 7.0 gradually.

Adjust the aforementioned treated filtrate to be isotonic and fill it into the 500 ml sealed flasks, then sterilize for 20 minutes at 0.1 MPa to obtain 400 kg medium. Its total volume is about 400,000 ml.

Recover the strain of S. aureus CGMCC 0485 at 35° C. for 24 hrs, then proliferate cells on the blood plates for 8 hrs at 35° C. to obtain the seed solution. The bacterium concentration of the seed solution is 105.

Then, mix the medium in a stainless steel vessel and put it into a fermenter after sterilizing and filtering. Inoculate it after preheating to 35° C. and the amount of inoculation is 0.02 ml per 100 ml medium. Ferment it at 35° C. for 20 hrs to obtain the culture. Fill it to the ampoule directly after the said culture is sterilized, filtered and examined eligibly.

Each culture obtained in fermentation is tested by pyrogen, enzyme activity and hypersusceptibility.

### EXPERIMENTAL EXAMPLE 1

### Test of Leucopenia Caused by Anti-Chemotherapy

The clinic test of leucopenia caused by anti-chemotherapy with the culture of S. aureus obtained in the example 1 of the present invention was implemented in Chinese-Japanese Friendship Hospital. Twenty (20) cases selected were mainly cancers treated by chemotherapy, which the most were lung cancer (above 60%). It was identified with tests of pathology and cytology that there were no effects to patients' livers and kidneys before and after muscle-injecting the culture of the present invention. It had distinct effects on reducing leucopenia caused by chemotherapy (p<0.05 and p<0.01). The effective rate on leucopenia caused by anti-chemotherapy was 90% in the period of treatment, the apparent rate was 55%, whereas the effective rate of the control group was only 15%, and the apparent rate was 5%.

Accordingly, the culture produced in the present invention had the effect on antagonizing leucopenia caused by anti-chemotherapy, protecting leucocytes not to decline or reducing the degree of leucopenia, shortening the period of leucopenia, and improving the declined cells to recovery.

### EXPERIMENTAL EXAMPLE 2

### The Effect on Natural Killing Cell Activity

It is reported from the test of Academy of Military Medical Sciences that the injection is prepared with the said culture of the present invention, and the injection comprised 500 u per ml. One unit of activity(u) herein is defined as the amount of free coagulase in 1 ml injection that releases 1 it g fibrin from liquid fibrinogen in plasm at 37° C. in 6 hrs. The tested tumors were: S180 sarcoma, Lewis lung cancer and U14 cervical carcinoma. The well-known method in the art was applied to test Kunming mice and C57BL/6 mice, the natural killing cell activity (NK) was assayed and the lymphocytes transformation experiment was performed. The results was as follows:

Two days after the culture of the present invention was injected, the NK activity increased, and it reached maximum 4 days later, then it came back to the level before drug was used gradually. The rate of cancer-suppress was above 90%.

After the drug was used, the change of mice with S180 sarcoma was as follows: NK activity could increase slightly 200-100 u per mouse. When the dosage was up to 1200-1500 u per mouse, NK activity increased distinctly. The NK activity also increased (p<0.05) distinctly after 32 u per day per time was used to mice with Lewis and mice with U14 cevix cancer for nine days.

Accordingly, injection of the culture produced by the present invention to mice one time or many times can increase the NK cell activity in normal mice and mice with Lewis lung carcinoma distinctly.

### EXPERIMENTAL EXAMPLE 3

### The Effect on the Transformation Rate of Lymphocyte

It is reported from the test of Academy of Military Medical Sciences that the E transformation rate of lymphocyte could increase slightly in 4 days, and they came back to normal 6 days later when the drugs of 32.5 u were used to 10 normal mice.

When the culture of the example 1 in the present invention was abdominal injected to mice with carcinoma, the transformation rate of lymphocyte could increase slightly in 9 days. When a high dosage (1000 or 1500 u) was used once, the transformation rate of lymphocyte could increase distinctly.

### EXPERIMENTAL EXAMPLE 4

### The suppressive Effect on Tumor Cells

It is reported from the test of Academy of Military Medical Sciences that extracted the ascites from mice with S180 ascites tumor and counted the number of tumor cells, then diluted to expected tumor cells. Added the different amount of the culture of example 1 in the present invention. Mixed them and inoculated 0.2 ml to each mouse. Killed the mouse and extracted and weighted the tumors 10 days later. The control groups did not contain the culture of the present invention, and just added the same volume of physiological salt solution. The rest was the same as that of the test group. The results showed that the dosage of 50 u per mouse had the distinct suppressive effect on the growth of S180 tumor compared with the control group; the average cancer-suppressive rate was 38.3±20.9%. The cancer-suppressive effect increased with the increase of dosage. The cancer-suppressive rate could be 91% if the used dosage was 200-1500 u.

### EXPERIMENTAL EXAMPLE 5

### The Treatment Effect on S180 Entitative Tumor

It is reported from the test of Academy of Military Medical Sciences that 86 mice were divided into 4 groups and the control group consisted of 23 mice, the other were 21 mice per group. Subcutaneous inoculated S180 ascites tumor cells to mice of test groups and injected the culture of example 2 in the present invention 24 hr later. One time per day for 9 days. Then killed the mice and extracted and weighted the tumors 10 days later. Whereas the control group were injected physiological salt solution. The results showed that the suppressive rate of S180 with the culture of 50, 100 and 150 u per mouse were 25%, 30% and 37% respectively.

Furthermore, The culture of the present invention was subcutaneous injected after C57BL/6 mice were subcutaneous inoculated the tumor cells 24 hr later, one time per day for 9 days; the dosage was 50, 100 and 150 u per mouse. The results showed that it had the slight suppressive effect.

### EXPERIMENTAL EXAMPLE 6

### The Result of Observation on Patients

With a great deal of clinic tests in Chinese Japanese Friendship Hospital, Qingdao People Hospital, The Fifth Hospital in Shenyang, Teaching Hospital, Bangbu Medical College, Teaching Hospital, Dalian Medical College and Tongren Hospital in Shanghai, including chemotherapy and actinotheraphy synthetical tests, and the effect on all aspects of patients of the present culture etc, these results showed that the culture of the present invention could increase the number of leucocytes reduced by chemotherapy and radiotherapy. Furthermore, the said culture used in the period of chemotherapy and actinotheraphy could prevent the decrease of leucocytes caused by chemotherapy and actinotheraphy. Due to the length, it is needless to say.

Furthermore, the culture of the present invention had the effects on reducing the toxic side effect in chemotherapy and actinotheraphy (such as marrow suppression, gastrointestinal tract reaction, inappetency, lose weight and activity etc.)

The culture of the present invention was safe. A few patients showed fever after the experiment began in 1-3 days, and the body temperature was about 38 centigrade, but they could improve by treatment. Most testees could tolerate and recover themselves or by slight treatment.

In a word, the evaluation result of synthetic treatment was that the apparent effect was 25.93%, the effective effect was 55.09%, the improving effect was 15.74% and the inefficiency was 3.24% respectively, so the total effective rate (apparent effect plus effective effect) was 81.02%.

What is claimed is:

1. A method of preparing the culture of Staphylococcus aureus, the method comprising:
1) adding a pork heart into 1-2 volume water, smashing, filtering and obtaining a first filtrate and residue;
2) adding 1-2 volume clean water of 90-95° C. into the residue, soaking, smashing and obtaining a second filtrate and residue; combining the first and second filtrates to obtain a third filtrate;
3) to the combined third filtrate, adding 0.025-0.5 g peptone and 0.3-0.9 g NaCl based on 100 ml resulting medium, then adding activated carbon, and adjusting pH from 8.5 to about 7.2-7.4 gradually to obtain resulting medium;
4) recovering and proliferating the strain of Staphylococcus aureus CGMCC 0485, and preparing the seed solution, inoculating it to the medium when its bacterium concentration is 10⁵-10⁷, and the amount of inoculation is 0.02-0.2 ml based on 100 ml resulting medium;
5) culturing the strain CGMCC 0485 at about 35° C. for about 15-20 hrs to obtain the culture;
6) sterilizing and filtering the said culture, adjusting the osmotic pressure to isotonic and pH to 6.8-7.4.

2. A method according to claim 1, wherein the said medium comprising 0.025-0.5 g peptone and 0.3-0.9 g NaCl based on 100 ml medium.

3. The culture of S. aureus prepared according to the method of claim 1.

4. Use of the culture of S. aureus according to claim 3 for producing a medicament used for the treatment of leucopenia caused in chemotherapy.

5. Use of the culture of S. aureus according to claim 3 for producing a medicament of antitumor.

6. A strain of Staphylococcus aureus, which was deposited at the China General Microorganism Collection Center (CGMCC) on Sep. 14, 2000, under the accession number of CGMCC 0485.

7. A method of preparing the culture of Staphylococcus aureus, the method comprising:
extracting pork heart tissue with water to obtain an aqueous filtrate;
combining at least a portion of the filtrate with peptone and NaCl to obtain an initial medium;
adding activated carbon to the initial medium and adjusting the pH thereof to obtain a resulting medium;
proliferating a strain of Staphylococcus aureus CGMCC 0485, so as to form a seed solution;
inoculating the resulting medium with the seed solution; and
culturing the resulting medium inoculated with the seed solution to obtain a culture.

8. A method according to claim 7, wherein the initial medium comprises 0.025-0.5 g peptone and 0.3-0.9 g NaCl based on 100 ml of initial medium.

9. A method according to claim 7, further comprising sterilizing and filtering the culture.

10. A culture of Staphylococcus aureus prepared according to the method comprising:
extracting pork heart tissue with water to obtain an aqueous filtrate;
combining at least a portion of the filtrate with peptone and NaCl to obtain an initial medium;
adding activated carbon to the initial medium and adjusting the pH thereof to obtain a resulting medium;
proliferating a strain of Staphylococcus aureus, so as to form a seed solution;
inoculating the resulting medium with the seed solution; and
culturing the resulting medium inoculated with the seed solution to obtain a culture.

11. The culture according to claim 10, wherein the filtrate of the culture is administered to a human in an effective amount so as to result in the treatment of leucopenia caused in chemotherapy.

12. The culture according to claim 10, wherein the filtrate of the culture is administered to a human in an effective amount so as to result in the treatment of a tumor.
